(19) United States
(12) Patent Application Publication
(10) Pub. No.: US 2023/0181211 A1
(43) Pub. Date: Jun. 15, 2023

(54) MINIMALLY-INVASIVE SURGICAL CUTTING DEVICES FOR CUTTING FIBROTIC TISSUE

(71) Applicant: TEXAS HEART INSTITUTE, Houston, TX (US)

(72) Inventors: Skylar J. Buchan, Houston, TX (US); Mathews M. John, Houston, TX (US); Allison Davis Post, Houston, TX (US); Mehdi Razavi, Houston, TX (US); Drew Bernard, Houston, TX (US); Jayachandrika Jayavasudevan, Katy, TX (US)

(73) Assignee: TEXAS HEART INSTITUTE, Houston, TX (US)

(21) Appl. No.: 18/077,335

(22) Filed: Dec. 8, 2022

Related U.S. Application Data
(60) Provisional application No. 63/287,576, filed on Dec. 9, 2021.

(51) Int. Cl. A61B 17/32

(52) U.S. Cl. CPC A61B 17/32002; A61B 2017/00398

(57) ABSTRACT

A surgical cutting device for performing a minimally-invasive surgical procedure and which includes an external hub including a housing and a cutter actuator housed within the housing, wherein the cutter actuator, a catheter including a first end coupled to the external hub, a second end opposite the first end, and an internal passage, a cutting tool coupled to the second end of the catheter and including a cutting element moveable relative to the second end of the catheter by the cutter actuator, and a motion transfer assembly extending through the internal passage of the catheter from the cutter actuator to the cutting tool, wherein the motion transfer assembly is configured to transfer motion from the cutter actuator to the cutting element of the cutting tool in response to the activation of the cutter actuator.

### CROSS-REFERENCE TO RELATED APPLICATIONS

This application claims benefit of U.S. provisional patent application Ser. No. 63/287,576 filed Dec. 9, 2021, and entitled “Minimally-Invasive Surgical Cutting Devices for Cutting Fibrotic Tissue,” which is hereby incorporated herein by reference in its entirety for all purposes.

### STATEMENT REGARDING FEDERALLY SPONSORED RESEARCH OR DEVELOPMENT

Not applicable.

### BACKGROUND

Organic tissue of a patient may experience injury or trauma during surgical intervention. In some instances, fibrotic scar tissue may form as a result of injury or trauma experienced by the organic tissue which may adhere different tissue planes of the organic tissue together in a process which may be referred to as fibrotic attachment. As one example, in some instances following a surgical intervention to a patient's heart, fibrotic tissue may form within the patient's heart at least partially adhering the epicardium to the pericardium, a condition known as pericardial adhesion. Future surgical interventions into the space occupied by a fibrotic attachment may be made significantly more difficult as the relatively tough, inflexible fibrotic tissue forming the fibrotic attachment may limit access by catheters, wires, and/or other minimally-invasive surgical tools, potentially necessitating a more invasive procedure. Additionally, fibrotic attachment may also endanger the patient by constricting or limiting organ function.

### BRIEF SUMMARY OF THE DISCLOSURE

An embodiment of a surgical cutting device for performing a minimally-invasive surgical procedure comprising an external hub comprising a housing and a cutter actuator housed within the housing, wherein the cutter actuator, a catheter comprising a first end coupled to the external hub, a second end opposite the first end, and an internal passage, a cutting tool coupled to the second end of the catheter and comprising a cutting element moveable relative to the second end of the catheter by the cutter actuator, and a motion transfer assembly extending through the internal passage of the catheter from the cutter actuator to the cutting tool, wherein the motion transfer assembly is configured to transfer motion from the cutter actuator to the cutting element of the cutting tool in response to the activation of the cutter actuator. In some embodiments, the external hub comprises a power supply housed within the housing, wherein the power supply powers the cutter actuator. In some embodiments, the cutter actuator comprises an output shaft configured to rotate about a rotational axis, and an actuator connector coupled to the output shaft and rotatable about the rotational axis, wherein the motion transfer assembly couples to the output shaft through the actuator connector. In some embodiments, the cutter actuator comprises an output shaft configured to rotate about a rotational axis, and a slider coupled to the output shaft and configured to reciprocate in opposed linear directions in response to rotation of the output shaft about the rotational axis. In certain embodiments, the motion transfer assembly comprises an actuator cable having a first end connected to the cutter actuator and a second end connected to the cutting tool. In certain embodiments, the cutter actuator comprises an output shaft configured to rotate about a rotational axis, and wherein the cutter actuator is configured to transport the actuator cable through the internal passage of the catheter in response to rotation of the output shaft about the rotational axis. In some embodiments, the cutting tool comprises a stationary cutting element, a moveable cutting element, and a stop configured to delimit a maximum amount of relative movement between the stationary cutting element and the moveable cutting element. In some embodiments, the cutting tool comprises a stationary cutting element and a moveable cutting element, and wherein the cutter actuator is configured to induce a repeating motion in the moveable cutting element relative to the stationary cutting element. In certain embodiments, the cutting tool comprises a stationary cutting element and a moveable cutting element pivotably connected to the stationary cutting element, and wherein the cutter actuator is configured to induce a repeating rocking motion in the moveable cutting element relative to the stationary cutting element.

An embodiment of a surgical cutting device for performing a minimally-invasive surgical procedure comprises an external hub, a catheter comprising a first end coupled to the external hub, and a second end opposite the first end, a cutting tool coupled to the second end of the catheter and comprising a stationary cutting element and a moveable cutting element, and a cutter actuator coupled to the cutting tool and configured to induce a repeating motion in the moveable cutting element relative to the stationary cutting element. In some embodiments, the stationary cutting element and moveable cutting element each comprise a base and a plurality of serrations extending from the base, and wherein the cutter actuator is configured to displace the moveable cutting element in opposed rotational directions about a cutting axis. In some embodiments, the cutting tool comprises a stop configured to delimit a maximum amount of relative movement between the stationary cutting element and the moveable cutting element. In some embodiments, the surgical cutting device comprises a cutter guard positioned about the second end of the catheter, the cutter guard comprising a deployed position that covers the cutting tool and a retracted position that exposes the cutting tool and is spaced from the deployed position relative to the second end of the catheter. In certain embodiments, the cutting tool comprises one or more sensors configured to determine an impedance of a tissue contacted by the cutting tool. In certain embodiments, the surgical cutting device comprises an electronic system coupled to the external hub and configured to classify the tissue contacted by the cutting tool based on impedance data provided by the one or more sensors. In some embodiments, the surgical cutting device comprises a motion transfer assembly extending through an internal passage of the catheter from the cutter actuator housed within a housing of the external hub to the cutting tool, wherein the motion transfer assembly is configured to transfer motion from the cutter actuator to the cutting element of the cutting tool in response to the activation of the cutter actuator. In certain embodiments, the moveable cutting element comprises a pivot joint that pivotably couples the moveable cutting element to the stationary cutting element.

An embodiment of a surgical cutting device for performing a minimally-invasive surgical procedure comprises an external hub, a catheter comprising a first end coupled to the external hub, and a second end opposite the first end, a cutter actuator, and a cutting tool coupled to the second end of the catheter and to the cutter actuator, wherein the cutting tool comprises a stationary cutting element, a moveable cutting element, and a cutter actuator comprising a stepper motor configured to control a maximum amount of relative movement between the stationary cutting element and the moveable cutting element, wherein the moveable cutting element is moveable in response to the activation of the cutter actuator. In some embodiments, the stationary cutting element and moveable cutting element each comprise a base and a plurality of serrations extending from the base, and wherein the cutter actuator is configured to displace the moveable cutting element in opposed rotational directions about a cutting axis in response to activation of the stepper motor. In some embodiments, the surgical cutting device comprises a cutter deployment assembly coupled to the catheter and configured to retract the cutting tool into an internal receptacle of the catheter. In certain embodiments, the surgical cutting device comprises a motion transfer assembly extending through an internal passage of the catheter from the cutter actuator housed within a housing of the external hub to the cutting tool, wherein the motion transfer assembly is configured to transfer motion from the cutter actuator to the cutting element of the cutting tool in response to the activation of the cutter actuator. In some embodiments, the motion transfer assembly comprises an actuator cable having a first end connected to the cutter actuator and a second end connected to the cutting tool via a biasing element coupled between the actuator cable and the cutting tool.

### BRIEF DESCRIPTION OF THE DRAWINGS

For a detailed description of exemplary embodiments of the disclosure, reference will now be made to the accompanying drawings in which:

FIGS. 1-3 are schematic views of an exemplary minimally-invasive surgical procedure;

FIG. 4 is a schematic view of an embodiment of a minimally-invasive surgical cutting device;

FIG. 5 is a block diagram of an embodiment of an electronics system of the surgical cutting device of FIG. 4;

FIG. 6 is a perspective view of an embodiment of a cutter actuator of the surgical cutting device of FIG. 4;

FIG. 7 is a front view of another embodiment of a cutter actuator of the surgical cutting device of FIG. 4;

FIG. 8 is a top view of the cutter actuator of FIG. 7;

FIG. 9 is a top cross-sectional view of an embodiment of a catheter of the surgical cutting device of FIG. 4;

FIG. 10 is perspective view of the catheter of FIG. 9;

FIG. 11 is a top cross-sectional view of another embodiment of a catheter of the surgical cutting device of FIG. 4;

FIG. 12 is a partial side cross-sectional view of the catheter of FIG. 11;

FIG. 13 is perspective view of the catheter of FIG. 11;

FIGS. 14 and 15 are side cross-sectional views of an embodiment of a cutter guard of the surgical cutting device of FIG. 4;

FIG. 16 is a perspective view of an embodiment of a cutting tool of the surgical cutting device of FIG. 4;

FIG. 17 is a perspective view of another embodiment of a cutting tool of the surgical cutting device of FIG. 4;

FIG. 18 is a schematic view of another embodiment of a cutting tool of the surgical device of FIG. 4;

FIGS. 19 and 20 are side cross-sectional views of another embodiment of a minimally-invasive surgical cutting device; and

FIG. 21 is a block diagram of another embodiment of an electronics system of the surgical cutting device of FIG. 4.

### DETAILED DESCRIPTION OF THE DISCLOSED EMBODIMENTS

The following discussion is directed to various exemplary embodiments. However, one skilled in the art will understand that the examples disclosed herein have broad application, and that the discussion of any embodiment is meant only to be exemplary of that embodiment, and not intended to suggest that the scope of the disclosure, including the claims, is limited to that embodiment.

Certain terms are used throughout the following description and claims to refer to particular features or components. As one skilled in the art will appreciate, different persons may refer to the same feature or component by different names. This document does not intend to distinguish between components or features that differ in name but not function. The drawing figures are not necessarily to scale. Certain features and components herein may be shown exaggerated in scale or in somewhat schematic form and some details of conventional elements may not be shown in interest of clarity and conciseness.

In the following discussion and in the claims, the terms “including” and “comprising” are used in an open-ended fashion, and thus should be interpreted to mean “including, but not limited to . . . ” Also, the term “couple” or “couples” is intended to mean either an indirect or direct connection. Thus, if a first device couples to a second device, that connection may be through a direct connection, or through an indirect connection via other devices, components, and connections. In addition, as used herein, the terms “axial” and “axially” generally mean along or parallel to a central axis (e.g., central axis of a body or a port), while the terms “radial” and “radially” generally mean perpendicular to the central axis. For instance, an axial distance refers to a distance measured along or parallel to the central axis, and a radial distance means a distance measured perpendicular to the central axis.

As described above, fibrotic attachment in organic tissue of a patient due to, for example, injury or trauma occurring to the patient's organic tissue during a surgical intervention may limit future surgical access to the area affected by the fibrotic attachment. For example, the fibrotic attachment may prevent the insertion of catheters, wires, and/or other minimally-invasive surgical tools into the area affected by the fibrotic attachment. Additionally, fibrotic attachments typically cannot be addressed by conventionally available minimally-invasive surgical tools. Instead, in conventional practice a more invasive surgical procedure may be performed to provide the access required to remediate the fibrotic attachment using a conventional surgical cutting tool such as scissors and the like. Alternatively, a vascular or vessel dilator may be used in an attempt to remediate the fibrotic attachment without needing to resort to a more invasive surgical procedure exposing the patient to relatively greater risk. However, vascular dilators lack a cutting structure capable of cutting through the relatively tough fibrotic tissue, and thus are generally inadequate for successfully remediating fibrotic attachments.

Accordingly, embodiments described herein include surgical cutting devices for remediating fibrotic attachments as part of a minimally-invasive surgical procedure. Particularly, surgical cutting devices described herein include a cutting tool connected to an external hub by a catheter extending between the cutting tool and the external hub. The cutting tool and distal end of the catheter coupled thereto may be inserted into the body of a patient and guided to a location of interest therein proximal to a fibrotic attachment to be remediated by an operator of the surgical cutting device. The external hub remains external of the patient's body and houses a cutter actuator for driving a moveable cutting element of the cutting tool. The cutter actuator may be controlled by the operator of the surgical cutting device via a user input located on the external hub. Additionally, the surgical cutting device includes a motion transfer assembly which extends through the catheter and transfers motion outputted by the cutter actuator to the moveable cutting element of the cutting tool. The motion of the moveable cutting element allows the cutting tool to cut and penetrate into the fibrotic attachment whereby the fibrotic attachment may be remediated by the cutting movement of the moveable cutting element. Additionally, by transferring motion from the externally located external hub to the cutting tool insertable into the patient's body, the size of the cutting tool and catheter may be minimized so as to minimize the size of the opening in the patient's body required to insert the cutting tool into the patient's body, thereby minimizing the invasiveness of the surgical procedure.

Referring initially to FIGS. 1-3, an embodiment of a minimally-invasive surgical cutting device 50 for cutting fibrotic tissue is shown. Particularly, FIGS. 1-3 illustrate organic tissue 5 of a patient 1, the organic tissue 5 including a first tissue plane or layer 10 and a second tissue plane or layer 20. In this exemplary embodiment, the first tissue layer 10 comprises an epicardium while the second tissue layer 20 comprises a pericardium of the patient's 1 heart; however, it may be understood that reference to the pericardium and epicardium with respect to tissue layers 10, 20 is only for convenience and tissue layers 10, 20 may pertain to various types of organic tissues of the patient 1. For example, in another application the tissue layers 10, 20 may comprise a tissue plane of an organ and a tissue plane of an abdominal wall of the patient 1.

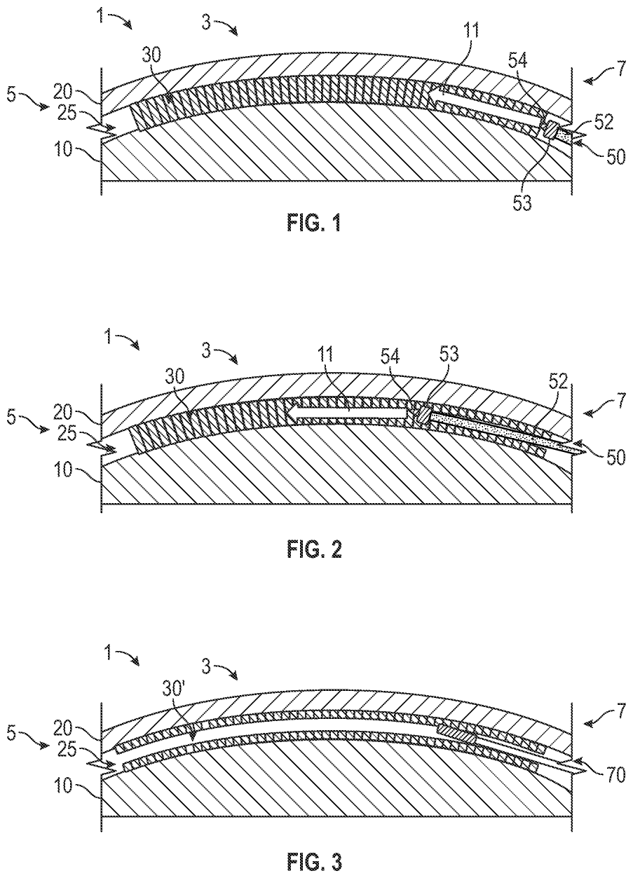

In a healthy patient, a cavity 25 (e.g., the pericardial cavity of the patient 1) is formed between the tissue layers 10, 20. However, in this instance, a fibrotic attachment has developed in the patient 1 between tissue layers 10, 20 in the form of fibrotic tissue (indicated generally by arrow 30 in FIGS. 1, 2) adhering the first tissue layer 10 to the second tissue layer 20. Fibrotic tissue 30 may have formed due to injury or trauma experienced by organic tissue 5 for various reasons including, for example, a previous surgical procedure. The fibrotic tissue 30 inhibits or prevents the insertion of catheters, wires, and/or other minimally-invasive surgical instruments into the cavity 25 which is now blocked by the fibrotic tissue 30.

The surgical cutting device 50 may cut into and through the fibrotic tissue 30 to thereby remediate the fibrotic attachment and free the first tissue layer 10 from the second tissue layer 20 as part of a minimally invasive surgical procedure. In this exemplary embodiment, the surgical cutting device 50 comprises a catheter 52 and an actuatable cutting tool 54 coupled to the catheter 52. Surgical cutting device 50 additionally includes an external hub coupled to the catheter 52 opposite cutting tool 54 but which is not shown in FIGS. 1-3. It may be understood that surgical cutting device 50 is shown in FIGS. 1-3 to illustrate how device 50 may be utilized in the remediation of a fibrotic attachment and the features of surgical cutting devices similar in configuration to surgical cutting device 50 will be discussed further herein.

In this exemplary embodiment, the catheter 52 extends between a first or proximal end coupled to the external hub and a second or distal end 53 connected to the cutting tool 54. The distal end of the catheter 52 and cutting tool 54 are each insertable into the patient's body 3 (the external hub and proximal end of catheter 52 remaining outside the patient's body 3) and may be guided towards location of interest 7 within the patient's body 3 proximal the fibrotic tissue 30. For example, a sheath or other tubular device may be inserted into the patient 1 which extends to the location of interest 7. The cutting tool 54 and catheter 52 may then be inserted through the pre-installed sheath to place the cutting tool 54 at the location of interest using a guidewire attached to the surgical cutting device 50. Alternatively, the catheter 52 may comprise a steerable catheter which may be directed towards the location of interest 7 by the user of surgical cutting device 50. Additionally, in some embodiments, signal markers may be placed on the catheter 52 and/or cutting tool 54 to identify the position of the catheter 52/cutting tool 54 within the patient's body 3. For example, one or more radiopaque markers comprising an X-ray absorbing material may be positioned on the catheter 52 and/or cutting tool 54 such that the position of the position of the catheter 52/cutting tool 54 is visible under fluoroscopic imaging.

The cutting tool 54 may be covered as the cutting tool 54 is inserted through the patient's body 3 towards the location of interest 7. For example, cutting tool 54 may be retracted into a body of the catheter 52 as each are inserted through the body 3 towards the location of interest 7. Alternatively, a separate guard may be deployed from the catheter 52 to cover the cutting tool 54 during transport of the cutting tool 54 to the location of interest 7. As shown particularly in FIG. 1, upon reaching the location of interest 7, the cutting tool 54 of surgical cutting device 50 is uncovered and exposed to the surrounding environment and a cutter actuator stored in the external hub is engaged to actuate the cutting tool 54 whereby the cutting tool 54 cuts into the fibrotic tissue 30.

The cutter actuator of surgical cutting device 50 may be powered by a power supply and/or the cutter actuator may be powered manually by the operator of surgical cutting device 50. An actuator transfer mechanism extending through catheter 52 transfers movement generated by the cutter actuator of the external hub to the cutting tool 54 to produce a repeating cutting motion in one or more cutting blades or elements of cutting tool 54. For example, the cutter actuator may produce a back-and-forth reciprocating motion in one or more cutting elements of the cutting tool 54. Alternatively, the cutter actuator may rotate one or more cutting elements of cutting tool 54 in one or more than one rotational direction about a rotational axis. Other forms of motion of the cutting tool 54 are also possible including, for example, a rocking, sliding, slicing, cleaving, or other motion.

As the cutting tool 54 is actuated by the cutter actuator of surgical cutting device 50, the cutting tool 54 is advanced (indicated by arrow 11 in FIGS. 1, 2) by the operator of surgical cutting device 50 through the fibrotic tissue 30 formed between tissue layers 10, 20, as shown particularly in FIGS. 1, 2. As described above, the position of cutting tool 54 and/or catheter 52 may be monitored by the operator of surgical cutting device 50 via signal markers may be placed on the catheter 52 and/or cutting tool 54. Additionally, in some embodiments, the surgical cutting device 50 may comprise one or more sensors to determine a position of the cutting tool 54, vital signs of the patient 1, and/or to classify or determine the type of tissue currently being cut by the cutting tool 54. For instance, the surgical cutting device 50 may comprise one or more electrodes for monitoring electrical impedance. The sensors of the surgical cutting device 50 may be utilized to visualize the catheter 52 and/or cutting tool 54 of surgical cutting device 50 using an electro-anatomical mapping system executing on, or in signal communication with, the external hub of surgical cutting device 50. Such electro-anatomical mapping systems include, for example, CARTO® 3 system (provided by Biosense Webster), EnSite® (provided by Abbott), and Rhythmia (provided by Boston Scientific).

The cutting tool 54 may continue to advance in the direction 11 until the entire length of fibrotic tissue has been cut (indicated by arrow 30′ in FIG. 3) by the cutting tool 54, thereby clearing or dislodging the fibrotic attachment formed between first tissue layer 10 from second tissue layer 20, as indicated in FIG. 3. The operator may determine that the fibrotic attachment has been remediated based on data provided by the sensors of surgical cutting device 50 described above. In some embodiments, the operator may determine the fibrotic attachment has been successfully remediated via the successful passage of a guidewire or other minimally invasive tool through the cavity 25 previously occupied by cut fibrotic tissue 30′ as confirmed by fluoroscopy or other imaging techniques. Following the removal of the fibrotic attachment, the cutting tool 54 may again be covered and the cutting tool 54 and catheter 52 may be withdrawn from the patient's body 3. With the cutting tool 54 and catheter 52 removed from the patient's body 3, a minimally-invasive surgical instrument 70 (shown in FIG. 3) may be inserted into the cavity 25 once occluded by the now cut fibrotic tissue 30′. A minimally-invasive surgical procedure now may be performed on the organic tissue 5 of the patient 1 which would not have been possible prior to the removal of the fibrotic attachment formed between tissue layers 10, 20.

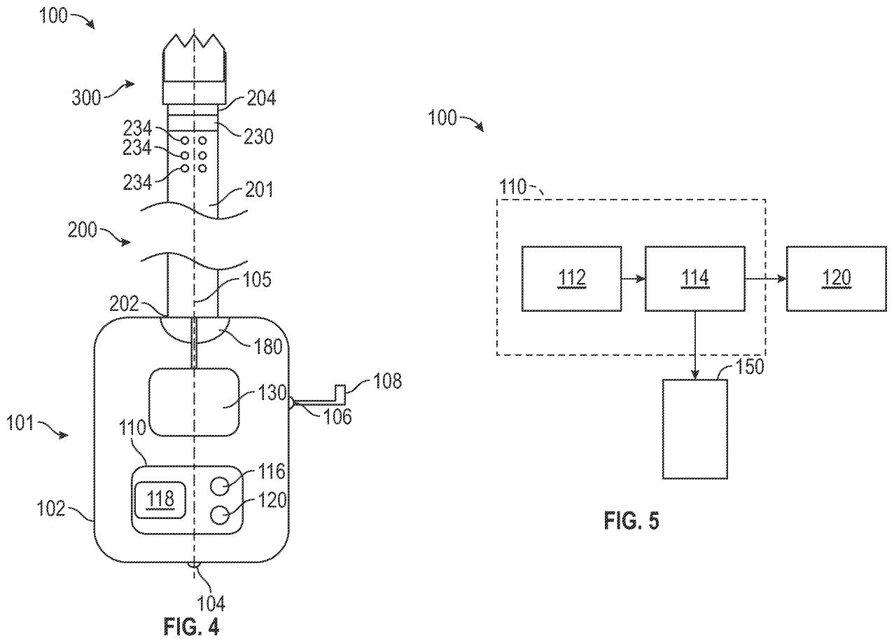

Referring now to FIGS. 4, 5 another embodiment of a minimally-invasive surgical cutting device 100 for removing a fibrotic attachment formed between a pair of tissues or tissue layers of a patient (e.g., the tissue layers 10, 20 of patient 1 shown in FIGS. 1-3). Surgical cutting device 100 may be operated in a manner similar to the operation of surgical cutting device 50 shown in FIGS. 1-3 in order to remove a fibrotic attachment from a patient as part of a minimally-invasive surgical procedure. In this exemplary embodiment, surgical cutting device 100 has a central or longitudinal axis 105 and includes an external hub 101, a catheter 200, and a cutting tool 300, where the catheter 200 extends between and couples with the external hub 101 and cutting tool 300. Similar to surgical cutting device 50 shown in FIGS. 1-3, the cutting tool 300 and a portion of the catheter 200 are insertable into the body of the patient while the external hub 101 remains external of the patient and is operated by a user of surgical cutting device 100.

In this exemplary embodiment, external hub 101 of surgical cutting device 100 generally includes an outer housing 102, an electronics system 110, a cutter actuator 130, and a deployment actuator 180. Housing 102 houses components of the electronics system 110, cutter actuator 130, and deployment actuator 180. In some embodiments, housing 102 is small and light enough to be hand-held by a single operator of surgical cutting device 100. In this manner, the operator of surgical cutting device 100 may, in some instances, manipulate the position of catheter 200 and/or cutting tool 300 by manually manipulating the position of the external hub 101 relative to the patient. Additionally, in this exemplary embodiment, housing 102 includes a port 104 for receiving a guidewire for transporting the cutting tool 300 and catheter 200 through a tubular structure (e.g., a sheath, etc.) previously installed in the body of the patient. However, it may be understood that in other embodiments housing 102 may not include guidewire port 104 including, for example, embodiments in which a steerable catheter 200 is employed.

Housing 102 of external hub 101 additionally includes a port 106 for receiving a manual actuator 108 (shown as a hand-crank in FIG. 4) for manually powering and operating the cutter actuator 130 of external hub 101. When desired, manual actuator 108 may be manually operated by a user of surgical cutting device 100 in lieu of electronics system 110. However, in other embodiments, housing 102 may not include port 106 and surgical cutting device 100 may not include manual actuator and instead the operation of cutter actuator 130 may only be powered by electronics system 110.

The electronics system 110 of external hub 101 is at least partially received in the housing 102 and, in this exemplary embodiment, provides a user interface for the operator of surgical cutting device 100 while also powering the actuators 130, 180 of external hub 101. As shown particularly in FIG. 5, in this exemplary embodiment, electronics system 110 includes a power source 112 and a control system 114 powered by the power source 112. In some embodiments, the power source 112 comprises a battery pack; however, it may be understood that the configuration of power source 112 may vary. The control system 114 controls the operation of the cutter actuator 130 and deployment actuator 180 of surgical cutting device 100, each of which are described further herein.

The control system 114 may control the operation of actuators 130, and/or 180 in response to control inputs provided by the operator of surgical cutting device 100. Particularly, in this exemplary embodiment, electronics system 110 additionally includes one or more user inputs 116, a visual display screen 118, and an electrical interface or port 120. The one or more user inputs 116 may comprise switches, dials, and/or joysticks for operating actuators 130, 180 and/or other components of surgical cutting device 100. For example, user inputs 116 may include a first switch for activating the cutting tool 300 via the cutter actuator 130 and/or for switching between electronic and manual (via manual actuator 108) operation of the cutter actuator 130. User inputs 116 may also include a second switch for deploying and retracting the cutting tool 300 via the deployment actuator 180. User inputs 116 may also include a toggle or joystick for operating a steering actuator (not shown in FIGS. 4, 5) of external hub 101 for steering the catheter 200 (e.g., along multiple independent axes) in embodiments in which catheter 200 comprises a steerable catheter.

The display screen 118 of electronics system 110 may display screen in real-time information pertaining to both surgical cutting device 100 and the patient. For example, display screen the operational status of cutting tool 300 such as if the cutting tool 300 is currently activated, and if it is currently retracted or deployed from catheter 200. Additionally, display screen 118 may indicate information based on data provided by one or more sensors of surgical cutting device 100. For example, display screen 118 may indicate information based on impedance data provided by one or more sensors of the surgical cutting device 100 and which may indicate to an operator of the surgical cutting device 100 a position of the cutting tool 300 and/or catheter 200 within the patient's body. The information indicated by display screen 118 based on the impedance data may also confirm whether the cutting tool 300 has reached a location of interest within the patient's body by classifying the type of tissue (based on the impedance data) currently engaged by the cutting tool 300. In some embodiments, display screen 118 may display screen an electrogram, such as an electrocardiogram, of the patient obtained by sensors of the surgical cutting device 100.

The electrical interface 120 of electronics system 110 permits the electronics system 110 to electrically connect with a second, external electronics system. For example, electronics system 110 may via electrical interface 120 connect with an external vital signal monitor, an electrical stimulator or other device for providing patient care, and/or an additional user interface for controlling one or more components (e.g., cutting tool 300) of surgical cutting device 100. Electrical interface 120 may also connect electronics system 110 with an external imaging system and/or electrophysiology recording system receiving data from the one or more sensors of surgical cutting device 100. Alternatively, electrical interface 120 may instead comprise a suction port from which debris formed by the operation of the cutting tool 300 may be removed from the patient's body.

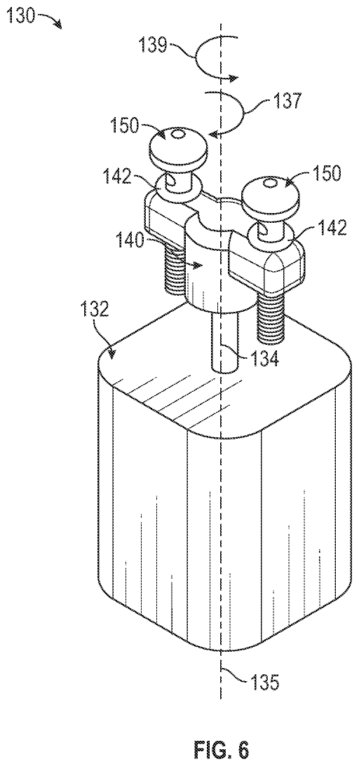

Referring now to FIG. 6, an embodiment of the cutter actuator 130 of surgical cutting device 100 is shown. As described above, cutter actuator 130 controls the operation of the cutting tool 300 of surgical cutting device 100. Particularly, cutter actuator 130 may, upon activation, induces a repeating cutting movement (e.g., reciprocating, pivoting, rotational, etc.) in one or more cutting elements or blades of the cutting tool 300. Cutter actuator 130 is housed within the housing 102 of external hub 101 and thus is maintained external to the patient's body during operation of surgical cutting device 100. As will be described further herein, the motion induced by cutter actuator 130 is transferred through catheter 200 to the cutting tool 300. In this manner, the size of the components of surgical cutting device 100 which are introduced into the patient's body during operation of surgical cutting device 100 is minimized to correspondingly minimize the invasiveness of the surgical procedure.

In this exemplary embodiment, cutter actuator 130, upon activation induces a repeating angular or rocking motion in one or more cutting elements of cutting tool 300. Particularly, in this exemplary embodiment, cutter actuator 130 generally includes an electric stepper motor 132, an actuator connector 140 coupled therewith, and a pair of tensioners 150. Stepper motor 132 is powered by the power source 112 of electronic system 110 of surgical cutting device 100 in this exemplary embodiment. In other embodiments, stepper motor 132 may be powered by a power source 112 that is external the hub 101 such as, for example, an electrical outlet. In this exemplary embodiment, upon activation by the control system 114 of electronic system 110, stepper motor 132 rotates an output shaft 134 connected thereto about a rotational axis 135. In this exemplary embodiment, rotational axis 135 extends substantially perpendicular to the central axis 105 (e.g., rotational axis 135 may extend out of the page in FIG. 4), but which may extend in alternative directions relative to central axis 105 in other embodiments.

Stepper motor 132 of cutter actuator 130 is generally configured to alternatingly rotate output shaft 134 in a first rotational direction (indicated by arrow 137 in FIG. 6) and a second rotational direction (indicated by arrow 139 in FIG. 6). It may be understood the rotary oscillation of output shaft 134 may be transferred through the actuator connector 140 to the cutting tool 300. The degree of rotation as well as the rate of rotation of output shaft 134 produced by stepper motor 132 in either rotational direction 137 and 139 may be controlled by the control system 114 of electronic system 110. For example, control system 114 may control or adjust the operation of stepper motor 132 to enhance the cutting ability of cutting tool 300.

The actuator connector 140 is coupled to a distal end of the output shaft 134 opposite stepper motor 132 and is configured to couple the output shaft 134 to a pair of actuator cables of the surgical cutting device 100 (as will be discussed further herein) whereby rotary motion of the output shaft 134 may be transferred to the cutting tool 300. In this exemplary embodiment, actuator connector 140 defines a pair of receptacles 142 which receive the pair of tensioners 150 of cutter actuator 130. Tensioners 150 are radially offset from the rotational axis 135 of cutter actuator 130 such that tensioners 150 revolve about the rotational axis 135 in response to the rotation of output shaft 134 by stepper motor 132. Additionally, tensioners 150 are coupled to actuator connector 140 via receptacles 142 whereby tensioners may be displaced longitudinally along axes parallel with rotational axis 135 to adjust the degree of tension in the actuator cables of the surgical cutting device 100. For example, tensioners 150 may be threadably received in receptacles 142 such that tensioners 150 may be threaded in either of a pair of opposed longitudinal directions extending along a given axis parallel with rotational axis 135. Additionally, in some embodiments, the actuator cables of surgical cutting device 100 may wrap around tensioners 150 to secure the cables with tensioners 150 whereby rotation of tensioners 150 about rotational axis 135 alternatingly pulls on the actuator cables 150. Alternatively, the actuator cables may be received in receptacles defined by tensioners 150 or otherwise coupled to tensioners 150 whereby motion applied to tensioners 150 from stepper motor 132 may be transferred to the actuator cables, and from the actuator cables to the cutting tool 300.

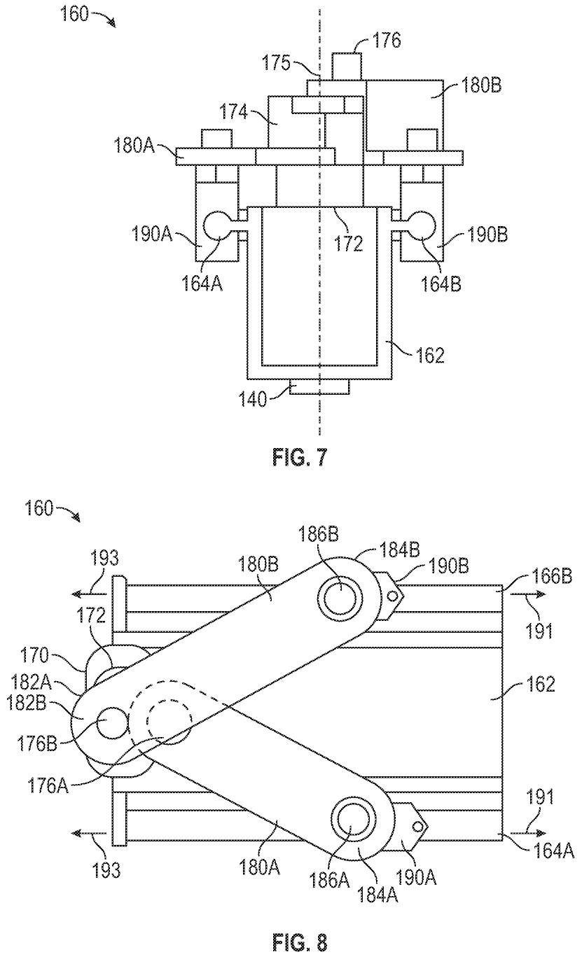

Referring to FIGS. 7 and 8, another embodiment of a cutter actuator 160 is shown. In some embodiments, surgical cutting device 100 may comprise cutter actuator 160 in lieu of the cutter actuator 130 shown in FIG. 6. In this exemplary embodiment, cutter actuator 160, upon activation, induces a repeating back-and-forth or reciprocating motion in one or more cutting elements of cutting tool 300. Particularly, in this exemplary embodiment, cutter actuator 160 generally includes a housing 162, an electric motor 170, a pair of connecting rods 180A, 180B, and a pair of linearly reciprocating sliders 190A, 190B. Housing 162 includes a pair of parallel extending linear tracks 164A, 164B to which the pair of sliders 190A, 190B, respectively, are coupled. Linear tracks 164A, 164B may each extend in directions orthogonal the central axis 105 of surgical cutting device 100; however, it may be understood that the orientation of cutter actuator 160 relative to central axis 105 of surgical cutting device 100 may vary.

In this exemplary embodiment, electric motor 170 is received within a receptacle of housing 162 and is powered by the power source 112 of electronic system 110 of surgical cutting device 100. In other embodiments, electric motor 170 may be powered by a power source 112 that is external the hub 101 such as, for example, an electrical outlet. In this exemplary embodiment, upon activation by the control system 114 of electronic system 110, electric motor 170 rotates an output shaft 172 connected thereto about a rotational axis 175 extending perpendicular to central axis 105 in this exemplary embodiment but which may extend in other directions relative to central axis 105 in other embodiments. Output shaft 172 is coupled to an eccentric shaft 174 having a pair of eccentric proximal pegs or pivot joints 176A, 1766, each of which are separately radially offset from rotational axis 175 and thus rotate eccentrically encircling the rotational axis 175 in response to rotation of output shaft 172 about the rotational axis 175. Although not shown in FIGS. 6, 7, cutter actuator 160 may include a clutch or other mechanism to interface the manual actuator 108 with output shaft 172 such that output shaft 172 may be manually rotated about rotational axis 175 via the manual actuator 108 when desired by an operator of surgical cutting device 100.

In this exemplary embodiment, each connecting rod 180A, 180B has a longitudinal first end 182A, 1826, respectively, and a longitudinal second end 184A, 1846, respectively, opposite first end 182A, 182B. The first end 182A of the first connecting rod 180A is pivotably coupled to the first pivot joint 176A of eccentric shaft 174 while the first end 1826 of the second connecting rod 1806 is pivotably coupled to the second pivot joint 1766 of eccentric shaft 174. Additionally, the second end 184A of the first connecting rod 180A is slidably coupled to the first slider 190A by a distal pivot joint 186A while the second end 1846 of the second connecting rod 1806 is slidably coupled to the slider 1906 by a distal pivot joint 1866. Further, the first slider 190A is slidably coupled to the first track 164A while the second slider 190B is slidably coupled to the second track 164B.

In the configuration described above, tracks 164A, 164B travel back-and-forth alternating between a first linear direction 191 and an opposing second linear direction 193 in response to rotation of the output shaft 172 about the rotational axis 175. Particularly, in this exemplary embodiment, eccentric shaft 174 and proximal pivot joints 176A, 1766 are arranged such that the linear motion of first slider 190A is always 180° out of phase with the motion of second slider 1906. To state in other words, sliders 190A, 1906 never travel in the same linear direction 191, 193. Instead, when first slider 190A travels in the first linear direction 191 the second slider 190B travels in the second linear direction 193 and vice versa. As will be described further herein, the alternating linear motion of sliders 190A, 1906 induces a corresponding repeating motion in one or more cutting elements of the cutting tool 300.

Referring now to FIGS. 4, 9, and 10, the catheter 200 transfers motion from the cutter actuator 130 of external hub 101 to the cutting tool 300. In this exemplary embodiment, catheter 200 comprises a tubular body 201 which extends along central axis between a longitudinal first or proximal end 202 and a longitudinally opposed second or distal end 204. The proximal end 202 of catheter 200 is connected to the housing 102 of external hub 101 while the distal end 204 of catheter 200 is connected to the cutting tool 300. The body 201 of catheter 200 may be formed from a semi-rigid material such as, for example, a copolymer.

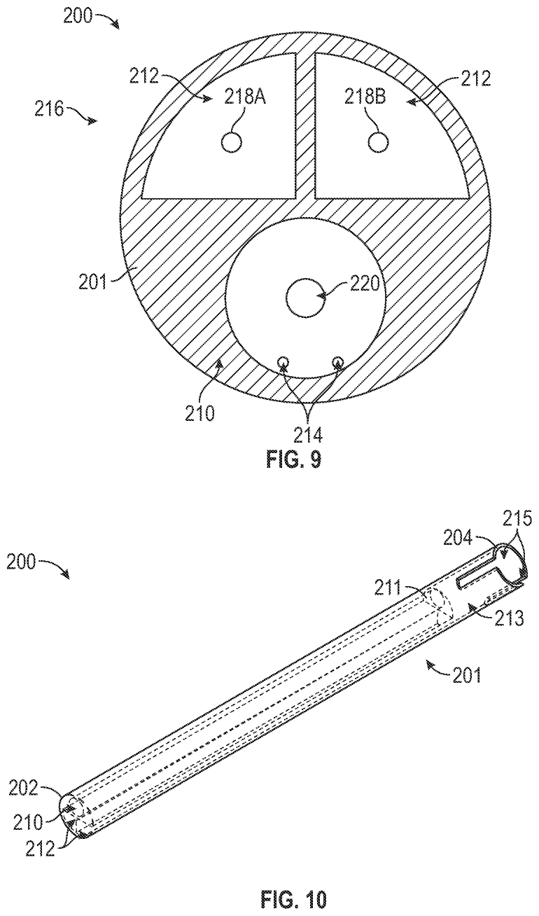

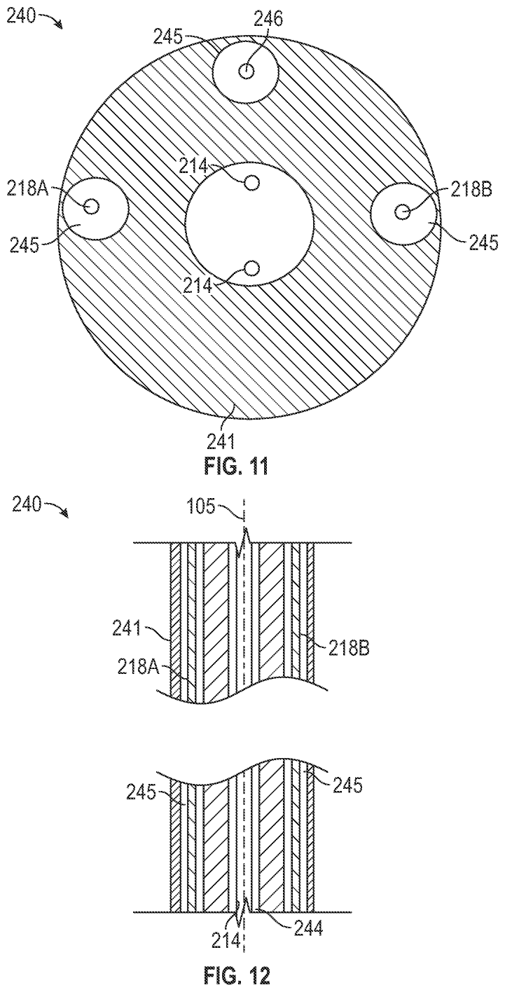

The body 201 of catheter 200 comprises a plurality of passages or lumens extending from proximal end 202 to the distal end 204. Particularly, in this exemplary embodiment, body 201 includes a communication passage 210 and a pair of actuator passages 212, and a common passage 213. While communication passage 210 is shown as generally cylindrical while actuator passages 212 are shown as pie-shaped in FIGS. 9 and 10, it may be understood that the geometry of passages 210, 212, and 213 may vary in other embodiments. In this exemplary embodiment, passages 210, 212 extend through body 201 from proximal end 202 to the common passage 213 while common passage 213 extends into body 201 from distal end 204. In this manner, passages 210, 212 are joined with common passage 213 at an interface 211 formed therebetween. In some embodiments, the portion of body 201 comprising common passage 213 may be formed separately and later joined to the portion of body 201 comprising passages 210, 212. In other embodiments, body 201 may be a singular member integral or monolithically formed via, for example, an injection molding process.

Additionally, in this exemplary embodiment, a pair of opposed radial slots 215 (spaced approximately 180° apart in this exemplary embodiment) extend into body 201 from distal end 204. As will be described further herein, cutting tool 300 may connect to the distal end 204 of catheter 200 via slots 215. Additionally, in other embodiments, common passage 213 may serve as a receptacle for cutting tool 300 when cutting tool 300 is retracted into the catheter 200 during transport of the cutting tool 300 through the patient's body to the location of interest. Thus, common passage 213 may also be referred to herein as receptacle 213. In still other embodiments, catheter 200 may not include either common passage 213 and/or slots 215. Additionally, the ends 202, 204 may be coupled to external hub 101 and cutting tool 300, respectively, via welding, gluing, other adhesion mechanisms, via one or more fasteners, and/or other coupling techniques.

In this exemplary embodiment, communication passage 210 includes a plurality of signal conductors or electrical cables 214. Although only a pair of electrical cables 214 are shown in FIG. 9, communication passage 210 may contain only a single electrical cable 214 or more than two electrical cables 214. Additionally, in other embodiments cables 214 may comprise signal conductors other than electrical cables such as, for example, fiber optic cables. Electrical cables 214 may transmit signals from sensors and/or electrodes of catheter 200 and/or cutting tool 300. Thus, some of the electrical cables 214 may extend entirely through communication passage 210 from the proximal end 202 to the distal end 204 of catheter 200 while other electrical cables 214 may only extend from the proximal end 202 of catheter 200 through a portion of the communication passage 210.

Catheter 200 additionally includes a motion transfer assembly 216 which, in this exemplary embodiment, extends through the pair of actuator passages of body 201 from the external hub 101 to the cutting tool 300. Motion transfer assembly 216 transfers the motion produced by cutter actuator 160 to the cutting tool 300 to thereby induce a corresponding motion in one or more cutting elements of the cutting tool 300. In this exemplary embodiment, motion transfer assembly 216 comprises a pair of flexible actuator cables 218A, 218B which extend through the corresponding pair of actuator passages 218 of body 201. In some embodiments, a first terminal end of a first actuator cable 218A connects to a first tensioner 150 of cutter actuator 130 while a first terminal end of a second actuator cable 218B connects to a second tensioner 150 of cutter actuator 130. In this configuration, rotation of the output shaft 134 of cutter actuator 130 results in the alternating pulling of actuator cables 218A, 2186 towards cutter actuator 130 and away from cutting tool 300.

For example, as output shaft 134 rotates about rotational axis 135 tension is applied to the first actuator cable 218A to pull the cable 218A towards cutter actuator 130 as tension is released from second actuator cable 218B, permitting cable 218B to travel towards cutting tool 300. As output shaft 134 continues to rotate, tension is released from the first actuator cable 218A, permitting cable 218A to travel towards cutting tool 300 as tension is applied by cutter actuator 130 to the second actuator cable 2186, pulling the second actuator cable 2186 towards cutter actuator 130. This process is repeated indefinitely until the activation of cutter actuator 130 (e.g., by the operator of surgical cutting device 100) is ceased. Thus, actuator cables 218A, 218B travel longitudinally through outer passages 212/common passage 213 in directions which may be parallel the central axis 105 of surgical cutting device 100. Additionally, while in this exemplary embodiment the motion transfer assembly 216 comprises actuator cables 218A, 218B, it may be understood that the configuration of motion transfer assembly 216 may vary in other embodiments. For example, in other embodiments, motion transfer assembly 216 may comprise a single elongate member or more than two elongate members. Additionally, in some embodiments, the first terminal end of first actuator cable 218A connects to the first slider 160A of the cutter actuator 160 shown in FIGS. 7 and 8 while the first terminal end of the second actuator cable 218B connects to the second slider 160B.

As shown particularly in FIG. 4, in this exemplary embodiment, catheter 200 additionally includes a signal marker 230 and a plurality of sensors 234. Signal marker 230 is located proximal the distal end 204 of catheter 200 and may be read by a signal detector to determine a position of the distal end 204 of catheter 200 within the patient's body. For example, signal marker 230 may comprise a radiopaque marker which may allow for the visualization of the distal end 204 of catheter 200 under fluoroscopic imaging. Sensors 234 may be positioned at different locations on catheter 200. For example, at least some of the sensors 234 may be located proximal to the distal end 204 of catheter 200 while others are spaced along the longitudinal length of catheter 200. In some embodiments, at least some of the sensors 234 comprise magnetic sensors which may be used to visualize the catheter 200 when inserted into the patient's body using, for example, an electro-anatomical mapping system (executing on the external hub 101 or on a computer system in signal communication with external hub 101).

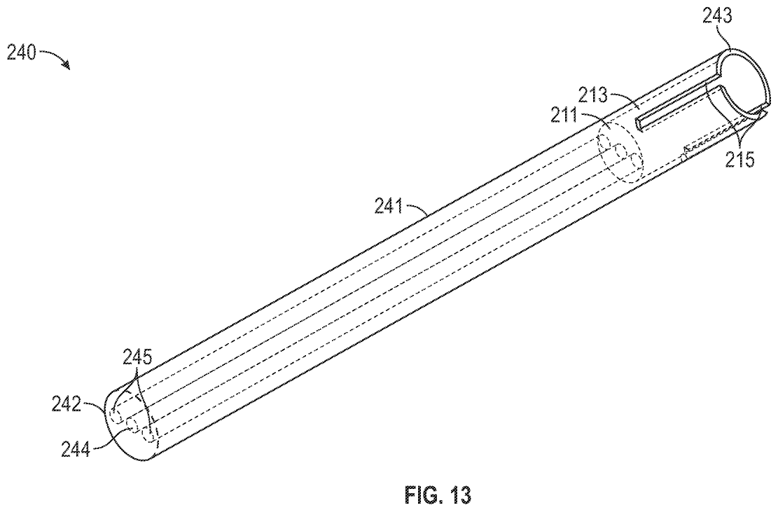

Referring now to FIGS. 11-13, another embodiment of a catheter 240 is shown. It may be understood that surgical cutting device 100 may comprise catheter 240 in lieu of the catheter 200 shown in FIGS. 9 and 10. Additionally, catheter 240 includes features in common with catheter 200, and shared features are labeled similarly. Catheter 240 comprises a tubular body 241 which extends along central axis between a longitudinal first or proximal end 242 and a longitudinally opposed second or distal end 243. Additionally, in this exemplary embodiment, body 241 includes a central communication passage 245 and a plurality pair of outer of offset passages 246, and the common passage 213. The pair of actuator cables 218A, 2186 extend through a pair of the outer passages 245 of body 241 while electrical cables 214 extend through the communication passage 244.

In addition to housing electrical cables 214, central passage 244 may also comprise a suction passage to which suction may be applied from external hub 101 whereby cuttings produced by cutting tool 300 may be removed from the patient's body via a port of the external hub 101. Further, in addition to motion transfer assembly 216, in this exemplary embodiment, one of the outer passages 212/common passage 213 of catheter 240 receives a guidewire 220 which extends through catheter 240 and may be used to guide cutting tool 300 and catheter 240 through the patient's body to a location of interest positioned therein. However, in other embodiments, catheter 240 may not include guidewire 220 such as, for example, in embodiments where catheter 240 comprises a steerable catheter controllable by the operator of surgical cutting device 100.

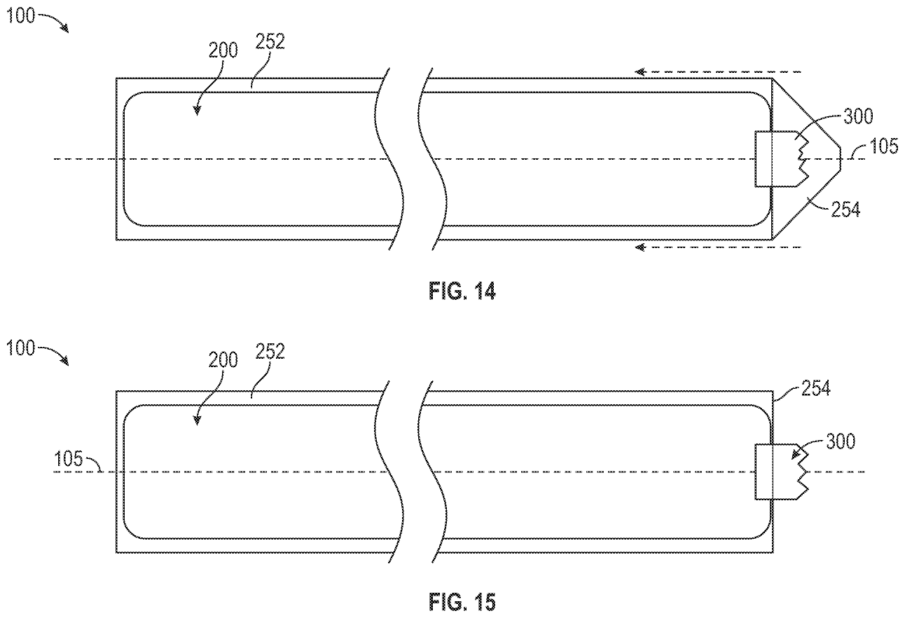

Referring now to FIGS. 14 and 15, an embodiment of a cutter guard 250 (hidden from view in FIG. 4) of the surgical cutting device 100 is shown. Cutter guard 250 covers the cutting tool 300 as cutting tool 300 is transported through the patient's body to the location of interest in the interest of preventing cutting tool 300 from inadvertently cutting or otherwise injuring healthy tissue of the patient. In this exemplary embodiment, cutter guard 250 comprises a tubular outer body or sleeve 252 and a conical tip 254 connected to and extending outwardly (along central axis 105) from the outer sleeve 252.

The outer sleeve 252 of cutter guard 250 is slidably positioned around the catheter 200 proximal the distal end 204 thereof. The cutter guard 250 is actuatable by the deployment actuator 180 of external hub 101 from an extended position (shown in FIG. 11) and a retracted position (shown in FIG. 12) by the 160 of external hub 101, where the extended position is spaced from the retracted position relative catheter 200 along the central axis 105 of surgical cutting device 100. In the extended position the tip 254 of cutter guard 250 is positioned over the cutting tool 300, preventing the cutting tool 300 from inadvertently cutting healthy tissue of the patient. However, in the retracted position, the cutting tool 300 is exposed form the cutter guard 250 and thus in a position to cut the fibrotic tissue forming the fibrotic attachment to be removed by surgical cutting device 100. The actuation of the cutter guard 250 between the retracted and extended positions may be controlled by the operator of the surgical cutting device 100 via one of the user inputs 116 of electronics system 110.

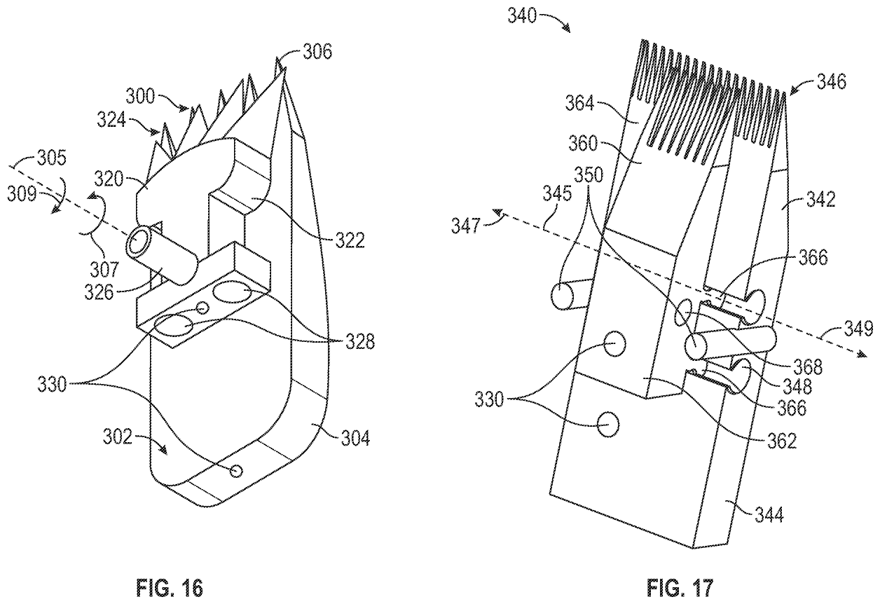

Referring now to FIG. 16, an embodiment of the cutting tool 300 of surgical cutting device 100 is shown. In this exemplary embodiment, cutting tool 300 generally includes a first or stationary blade 302 and a second or moveable blade 320 configured to travel back-and-forth in a rocking motion relative to the stationary blade 302 about a cutting axis 305 of the cutting tool 300. In this exemplary embodiment, the cutting axis 305 is orthogonal the longitudinal axis 105 of surgical cutting device 100; however, it may be understood that the orientation of cutting axis 305 relative to central axis 105 may vary in other embodiments.

The stationary blade 302 of cutting tool 300 includes a base 304, a stationary cutting element 306 in the form of a plurality of serrations or teeth opposite the base 304. In some embodiments, at least a portion of the base 304 of stationary blade 302 is received within the slots 215 of catheter 200 to couple the stationary blade 302 to the body 201 of catheter 200 whereby relative movement between stationary blade 302 and body 201 is restricted. Blades 302, 320 are formed from a rigid material. For example, blades 302, 320 may be formed from a metallic material (e.g., stainless steel, copper, etc.), rigid polymers such as, for example, polyethylene, polyether ether ketone (PEEK), ceramics, and/or plastics.

The moveable blade 320 of cutting tool 300 includes a base 322, a moveable cutting element 324 in the form of a corresponding plurality of serrations or teeth opposite the base 322, and a pivot joint or connector 326 extending along the cutting axis 305. In some embodiments, the tips of the teeth of cutting elements 306, 324 of blades 302, 320, respectively, may be blunted or protected by a wear resistant coaching while the edges of the teeth of cutting elements 306, 324 may be sharp for cutting into the fibrotic tissue of the patient.

In this exemplary embodiment, pivot joint 326 of moveable blade 320 pivotably couples the moveable blade 320 to the stationary blade 302 such that moveable blade 320 is permitted to pivot or rotate relative to the stationary blade 302 about the cutting axis 305 in either rotational direction. In some embodiments, the base 304 of stationary blade 302 may include a pair of oppositely positioned protrusions or stops flanking the moveable blade 320 and configured to permit a predefined degree of relative angular travel of the moveable blade 320 about the cutting axis 305 relative to the stationary blade 302. The back-and-forth relative motion of blades 302, 320 about cutting axis 305 results in a rocking motion which trims and cuts into fibrotic tissue to remove a fibrotic attachment formed by the fibrotic tissue. While in this exemplary embodiment cutting tool 300 provides a rocking motion to cut fibrotic tissue, it may be understood that in other embodiments cutting tools of surgical cutting device 100 may provide a rectilinear trimming motion, a rotary motion (e.g., a drilling motion), and other forms of cutting motions for cutting, drilling, tearing, and/or shearing fibrotic tissue of a patient.

In this exemplary embodiment, the motion transfer assembly 216 is connected to the moveable blade 320 of cutting tool 300 and displaces moveable blade 320 back-and-forth about cutting axis 305 in response to the activation of cutter actuator 130. Particularly, second terminal ends of actuator cables 218A, 218B (the first terminal ends of cables 218A, 2186 being connected to tensioners 150, respectively) connect to attachment points 328 of moveable blade 320 positioned on a proximal end (opposite cutting element 324) of the base 322 of moveable blade 320. In this configuration, in response to the activation of cutter actuator 130, first actuator cable 218A is pulled towards cutter actuator 130 by the rotational movement of the first tensioner 150, causing moveable blade 320 to travel in a first rotational direction 307 about the cutting axis 305. Subsequently, tension applied to the first actuator cable 218A from cutter actuator 130 is released and second actuator cable 2186 is pulled towards cutter actuator 130 by the rotational movement of the second tensioner 150, causing moveable blade 320 to travel in an opposed, second rotational direction 309 along cutting axis 305. The alternating movement of moveable blade 320 in opposed rotational directions 307, 309 along cutting axis 305 may be repeated indefinitely until cutter actuator 130 is deactivated by an operator of surgical cutting device 100.

Cutting tool 300 additionally includes sensors 330 in this exemplary embodiment for sensing phenomena of the patient during operation of the surgical cutting device 100 as part of a minimally-invasive surgical procedure. For example, sensors 330 may comprise electrodes configured to sense or determine an impedance of the tissue contacted by cutting tool 300. The type of tissue currently being cut by cutting tool 300 may be determined by electronic system 110 of surgical cutting device 100 based on the impedance data provided by sensors 330. Sensors 330 are electrically connected to the electronic system 110 of surgical cutting device 100 via the electrical cables 214 of catheter 200. In some embodiments, electronic system 110 may automatically disable cutting tool 300 and deactivate cutter actuator 130 based on data provided by sensors 330. For example, electronic system 110 may automatically disable cutting tool 300 in response to determining that cutting tool 300 is in contact with or cutting healthy, non-fibrotic tissue of the patient based on impedance data provided by sensors 330.

Although not shown in FIG. 16, cutting tool 300 may additionally include a debris wiper for clearing cut tissue and other debris from the cutting tool 300. Cutting tool 300 and/or catheter 200 may also include a corresponding suction device (e.g., a small pump or fan housed within the catheter 200, for example) which suctions the wiped debris from the cutting tool 300 and into and through the catheter 200 (e.g., through the central passage 210 of catheter 200) where the debris may be expelled external the patient's body via a suction port formed in the external hub 101 of surgical cutting device 100.

Referring now to FIG. 17, another embodiment of a cutting tool 340 is shown. It may be understood that surgical cutting device 100 may comprise cutting tool 340 in lieu of the cutting tool 300 shown in FIG. 16. Additionally, cutting tool 340 includes features in common with cutting tool 300, and shared features are labeled similarly. Cutting tool 340 comprises a first or stationary blade 342 and a second or moveable blade 360 configured to travel back-and-forth in a linear reciprocating motion relative to the stationary blade 360 along about a cutting axis 345 of the cutting tool 340.

The stationary blade 342 of cutting tool 340 includes a base 344, stationary cutting element 306, and a pair of linear tracks 348 formed in the base 344. The moveable blade 360 of cutting tool 340 includes a base 362, moveable cutting element 324, and a pair of rails 366 extending laterally from the base 362. The rails 366 of moveable blade 360 are slidably and interlockingly received within the tracks 348 of stationary blade 342 whereby a predefined degree of relative travel along cutting axis 345 is permitted while rotation and other forms of relative movement between blades 342, 360 is restricted. Particularly, stationary blade 342 additionally includes a pair of opposed stops or guide pillars 350 extending laterally from the base 344 of stationary blade 342, where the moveable blade 360 is positioned between the pair of stops 350. The width extending between stops 350 delimits or defines the amount of relative travel along cutting axis 345 between blades 342, 360 as contact between stops 350 and lateral sides of the base 362 of moveable blade 360 prevents further relative travel between blades 342, 360.

In this exemplary embodiment, the motion transfer assembly 216 displaces moveable blade 360 back-and-forth along cutting axis 345 in response to the activation of cutter actuator 130. Particularly, second terminal ends of actuator cables 218A, 218B (the first terminal ends of cables 218A, 218B being connected to tensioners 150, respectively) connect to attachment points 368 of moveable blade 360 positioned on lateral sides of the base 362 of moveable blade 360. In this configuration, in response to the activation of cutter actuator 130, first actuator cable 218A is pulled towards cutter actuator 130 by the rotational movement of the first tensioner 150, causing moveable blade 360 to travel in a first linear direction 347 along the cutting axis 345. Subsequently, tension applied to the first actuator cable 218A from cutter actuator 130 is released and second actuator cable 2186 is pulled towards cutter actuator 130 by the rotational movement of the second tensioner 150, causing moveable blade 360 to travel in an opposed, second linear direction 349 along cutting axis 345. The alternating movement of moveable blade 360 in opposed linear directions 347, 349 along cutting axis 345 may be repeated indefinitely until cutter actuator 130 is deactivated by an operator of surgical cutting device 100.

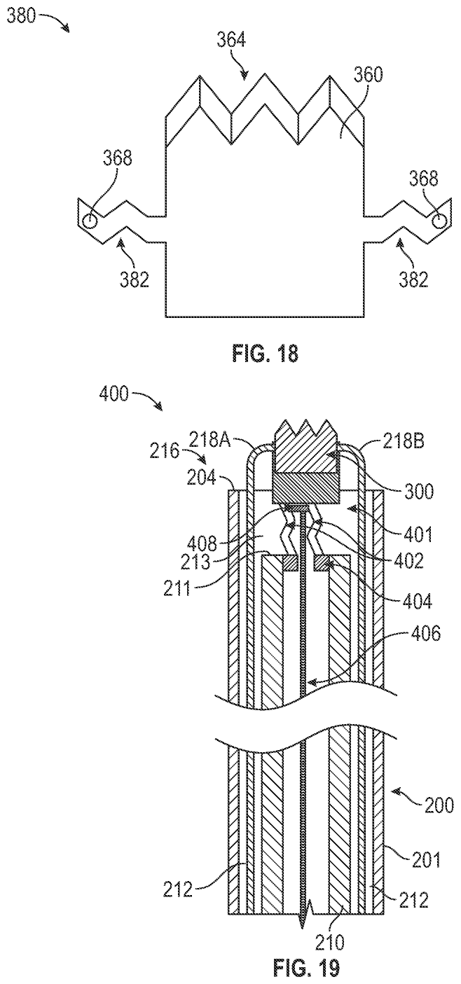

In some embodiments, one or more techniques may be employed to reduce the strain imparted to motion transfer assembly 216 during operation. For example, in some embodiments, actuator cables 218A, 218B may be coated with a lubricant to reduce friction between actuator cables 218A, 218B and catheter 200/external hub 101 during operation. In certain embodiments, a biasing element may be utilized to reduce strain in actuator cables 218A, 218B during operation. For example, referring briefly to FIG. 18, an embodiment of a cutting tool 380 is shown which is similar to cutting tool 340 but additionally includes a pair of strain relief elements in the form of biasing elements 382 connected between actuator cables 218A, 218B and the attachment points 368 of the moveable blade 360 of cutting tool 380. In this configuration, at least some of the tension applied to actuator cables 218A, 218B by cutter actuator 130 of the external hub 101 may be absorbed by biasing elements 382 to prevent actuator cables 218A, 218B from becoming damaged during operation.

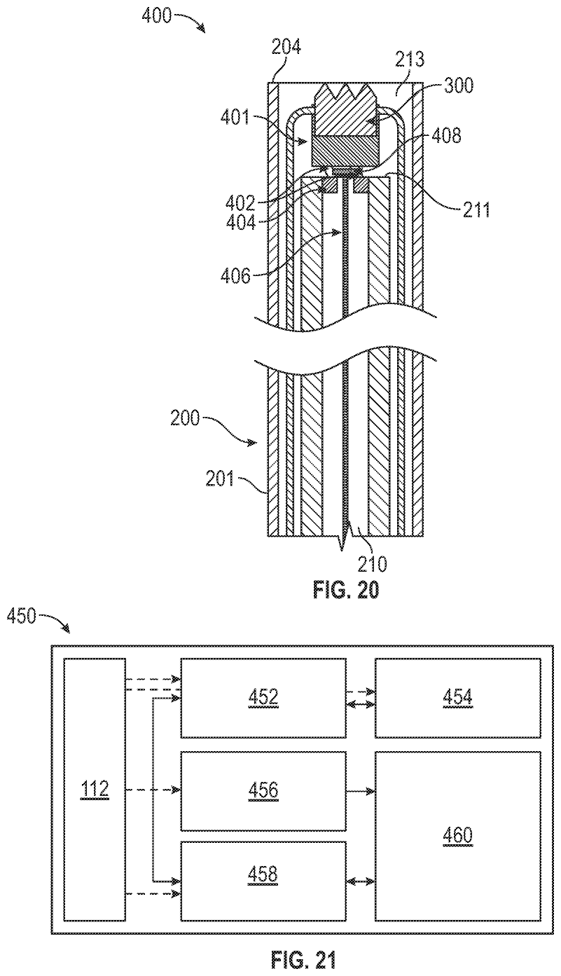

Referring now to FIGS. 19 and 20, another embodiment of a minimally-invasive surgical cutting device 400 is shown for removing a fibrotic attachment formed between a pair of tissues or tissue layers of a patient (e.g., the tissue layers 10, 20 of patient 1 shown in FIGS. 1-3). Surgical cutting device 400 includes features in common with the surgical cutting device 100 shown in FIG. 4, and shared features are labeled similarly. Surgical cutting device 400 is similar in configuration and operation as surgical cutting device 100 except that, instead of including cutter guard 250, the cutting tool 300 of surgical cutting device 400 is extendable from and retractable into the catheter 200 of surgical cutting device 400.

Particularly, in this exemplary embodiment, surgical cutting device 100 includes a cutter deployment assembly or deployer 401 which generally includes a biasing element 402 and an actuator cable 406. Biasing element 402 extends between the catheter 200 and cutting tool 300 and is configured to bias the cutting tool 300 outwardly from the distal end 204 of catheter 200. In this exemplary embodiment, a longitudinal first end of biasing element 402 is connected to an anchor 404 of the catheter 200 located at the interface 211 of catheter. Additionally, a longitudinal second end, opposite the first end, of biasing element 402 is connected to the cutting tool 300. For example, the second end of biasing element 402 may be connected to the base 304 of the stationary blade 302 of cutting tool 300.

The actuator cable 406 of cutter deployer 401 is connected to a retraction actuator of the external hub 101 of surgical cutting device 400 at a first terminal end thereof while an opposing second terminal end of the actuator cable 406 is connected to an anchor 408 coupled to cutting tool 300, such as, for example, to the base 304 of stationary blade 302. Additionally, actuator cable 406 extends through the central passage 210 of catheter 200. In this configuration, an operator of surgical cutting device 400 may activate the retraction actuator of external hub 101 (e.g., via one of the user inputs 116) to transport cutting tool 300 from a deployed position (shown in FIG. 19) external the distal end 204 of catheter 200 to a retracted position (shown in FIG. 20) in which the cutting tool 300 is received within the receptacle 213 of catheter 200. The retraction actuator of external hub 101 may retract cutting tool 300 by applying a tension to actuator cable 406 which overwhelms the biasing force applied to cutting tool 300 by biasing element 402 and thereby pulls cutting tool 300 towards and into the receptacle 213 of catheter 200.

Conversely, an operator of surgical cutting device 400 may deactivate the retraction actuator of external hub 101 to thereby release the tension applied to actuator cable 406. With tension released from actuator cable 406, biasing element 402 automatically transports the cutting tool from the retracted position to the deployed position.

Referring now to FIG. 21, another embodiment of an electronics system 450 of the surgical cutting device 100 of FIG. 4 is shown. Electronics system 450 is similar to the electronics system 110 shown in FIGS. 4, 5 but includes some features in addition to those included in electronics system 110. For example, in this exemplary embodiment, electronics system 450 includes a microcontroller 452, a communication unit 454 in signal communication with the microcontroller 452, an actuator controller 456 controlled by the microcontroller 452, a sensor unit 458 in signal communication with the microcontroller 452, and an external electrode interface 460 in signal communication with both the actuator controller 456 and sensor unit 458.

Actuator controller 456 controls the operation of one of the actuators of surgical cutting device 100. In this exemplary embodiment, actuator controller 456 controls the operation of cutter actuator 130; however, in other embodiments, actuator controller 456 may control the operation of other actuators including, for example, deployment actuator 180. The sensor unit 458 receives and/or processes sensor data from sensors of surgical cutting device 100 including, for example, sensors 234 of catheter 200 and/or sensors 330 of cutting tool 300. Additionally, sensor data is provided from sensor unit 458 to the microcontroller 452. The communication unit 454 permits electronic system 450 to communicate with computer systems or devices separate from external hub 101 of surgical cutting device 100. Additionally, the external electrode interface 460 allows for an operator of surgical cutting device 100 to account for real-time feedback of sense signals in the use of surgical cutting device 100. For example, impedance data captured by sensors 330 may be provided to the user to allow the user to identify different tissue planes (e.g., tissue layers 10, 20) in order to prevent the inadvertent cutting of tissue not intended to be cut by the user. Additionally, the cutting tool 300 may include voltage sensors which may be utilized to create a voltage map that is indicated to the user, eliminating the need to utilize a separate catheter or tool to create such a voltage map.

While embodiments of the disclosure have been shown and described, modifications thereof can be made by one skilled in the art without departing from the scope or teachings herein. The embodiments described herein are exemplary only and are not limiting. Many variations and modifications of the systems, apparatus, and processes described herein are possible and are within the scope of the disclosure. For example, the relative dimensions of various parts, the materials from which the various parts are made, and other parameters can be varied. Accordingly, the scope of protection is not limited to the embodiments described herein, but is only limited by the claims that follow, the scope of which shall include all equivalents of the subject matter of the claims. Unless expressly stated otherwise, the steps in a method claim may be performed in any order. The recitation of identifiers such as (a), (b), (c) or (1), (2), (3) before steps in a method claim are not intended to and do not specify a particular order to the steps, but rather are used to simplify subsequent reference to such steps.

What is claimed is:

1. A surgical cutting device for performing a minimally-invasive surgical procedure, the surgical cutting device comprising:
an external hub comprising a housing and a cutter actuator housed within the housing, wherein the cutter actuator;
a catheter comprising a first end coupled to the external hub, a second end opposite the first end, and an internal passage;
a cutting tool coupled to the second end of the catheter and comprising a cutting element moveable relative to the second end of the catheter by the cutter actuator; and
a motion transfer assembly extending through the internal passage of the catheter from the cutter actuator to the cutting tool, wherein the motion transfer assembly is configured to transfer motion from the cutter actuator to the cutting element of the cutting tool in response to the activation of the cutter actuator.

2. The surgical cutting device of claim 1, wherein the external hub comprises a power supply housed within the housing, wherein the power supply powers the cutter actuator.

3. The surgical cutting device of claim 1, wherein the cutter actuator comprises an output shaft configured to rotate about a rotational axis, and an actuator connector coupled to the output shaft and rotatable about the rotational axis, wherein the motion transfer assembly couples to the output shaft through the actuator connector.

4. The surgical cutting device of claim 1, wherein the cutter actuator comprises an output shaft configured to rotate about a rotational axis, and a slider coupled to the output shaft and configured to reciprocate in opposed linear directions in response to rotation of the output shaft about the rotational axis.

5. The surgical cutting device of claim 1, wherein the motion transfer assembly comprises an actuator cable having a first end connected to the cutter actuator and a second end connected to the cutting tool.

6. The surgical cutting device of claim 5, wherein the cutter actuator comprises an output shaft configured to rotate about a rotational axis, and wherein the cutter actuator is configured to transport the actuator cable through the internal passage of the catheter in response to rotation of the output shaft about the rotational axis.

7. The surgical cutting device of claim 1, wherein the cutting tool comprises a stationary cutting element, a moveable cutting element, and a stop configured to delimit a maximum amount of relative movement between the stationary cutting element and the moveable cutting element.

8. The surgical cutting device of claim 1, wherein the cutting tool comprises a stationary cutting element and a moveable cutting element, and wherein the cutter actuator is configured to induce a repeating motion in the moveable cutting element relative to the stationary cutting element.

9. The surgical cutting device of claim 1, wherein the cutting tool comprises a stationary cutting element and a moveable cutting element pivotably connected to the stationary cutting element, and wherein the cutter actuator is configured to induce a repeating rocking motion in the moveable cutting element relative to the stationary cutting element.

10. A surgical cutting device for performing a minimally-invasive surgical procedure, the surgical cutting device comprising:
an external hub;
a catheter comprising a first end coupled to the external hub, and a second end opposite the first end;
a cutting tool coupled to the second end of the catheter and comprising a stationary cutting element and a moveable cutting element; and
a cutter actuator coupled to the cutting tool and configured to induce a repeating motion in the moveable cutting element relative to the stationary cutting element.

11. The surgical cutting device of claim 10, wherein the stationary cutting element and moveable cutting element each comprise a base and a plurality of serrations extending from the base, and wherein the cutter actuator is configured to displace the moveable cutting element in opposed rotational directions about a cutting axis.

12. The surgical cutting device of claim 10, wherein the cutting tool comprises a stop configured to delimit a maximum amount of relative movement between the stationary cutting element and the moveable cutting element.

13. The surgical cutting device of claim 10, further comprising a cutter guard positioned about the second end of the catheter, the cutter guard comprising a deployed position that covers the cutting tool and a retracted position that exposes the cutting tool and is spaced from the deployed position relative to the second end of the catheter.

14. The surgical cutting device of claim 10, wherein the cutting tool comprises one or more sensors configured to determine an impedance of a tissue contacted by the cutting tool.

15. The surgical cutting device of claim 14, further comprising an electronic system coupled to the external hub and configured to classify the tissue contacted by the cutting tool based on impedance data provided by the one or more sensors.

16. The surgical cutting device of claim 10, further comprising a motion transfer assembly extending through an internal passage of the catheter from the cutter actuator housed within a housing of the external hub to the cutting tool, wherein the motion transfer assembly is configured to transfer motion from the cutter actuator to the cutting element of the cutting tool in response to the activation of the cutter actuator.

17. The surgical cutting device of claim 10, wherein the moveable cutting element comprises a pivot joint that pivotably couples the moveable cutting element to the stationary cutting element.

18. A surgical cutting device for performing a minimally-invasive surgical procedure, the surgical cutting device comprising:
an external hub;
a catheter comprising a first end coupled to the external hub, and a second end opposite the first end;
a cutter actuator; and
a cutting tool coupled to the second end of the catheter and to the cutter actuator, wherein the cutting tool comprises a stationary cutting element, a moveable cutting element, and a cutter actuator comprising a stepper motor configured to control a maximum amount of relative movement between the stationary cutting element and the moveable cutting element, wherein the moveable cutting element is moveable in response to the activation of the cutter actuator.

19. The surgical cutting device of claim 18, wherein the stationary cutting element and moveable cutting element each comprise a base and a plurality of serrations extending from the base, and wherein the cutter actuator is configured to displace the moveable cutting element in opposed rotational directions about a cutting axis in response to activation of the stepper motor.

20. The surgical cutting device of claim 18, further comprising a cutter deployment assembly coupled to the catheter and configured to retract the cutting tool into an internal receptacle of the catheter.

21. The surgical cutting device of claim 18, further comprising a motion transfer assembly extending through an internal passage of the catheter from the cutter actuator housed within a housing of the external hub to the cutting tool, wherein the motion transfer assembly is configured to transfer motion from the cutter actuator to the cutting element of the cutting tool in response to the activation of the cutter actuator.

22. The surgical cutting device of claim 21, wherein the motion transfer assembly comprises an actuator cable having a first end connected to the cutter actuator and a second end connected to the cutting tool via a biasing element coupled between the actuator cable and the cutting tool.
